(19) United States
(12) Patent Application Publication
(10) Pub. No.: US 2006/0275882 A1
(43) Pub. Date: Dec. 7, 2006

(54) Mash viscosity reduction

(75) Inventors: Ramiro Martinez-Gutierrez, Madrid (ES); Alain Destexhe, Kuringen (BE); Hans Sejr Olsen, Holte (DK); Marcel Mischler, Himmelried (CH)

Correspondence Address: NOVOZYMES NORTH AMERICA, INC., 500 FIFTH AVENUE, SUITE 1600, NEW YORK, NY, 10110, US

(73) Assignee: Novozymes, Bagsvaerd (DK)

(21) Appl. No.: 10/558,628

(22) PCT Filed: Apr. 2, 2004
(86) PCT No.: PCT/DK04/00231

§ 371 Date: Jul. 13, 2006

(30) Foreign Application Priority Data
Apr. 4, 2003 (DK) PA 2003 00517

(51) Int. Cl. C12P 7/06; C12P 19/02

(52) U.S. Cl. 435/161; 435/105

(57) ABSTRACT

The invention relates to a process for producing a fermentation product wherein the viscosity of the mash is reduced by application of beta-glucanase and xylanase activity.

### FIELD OF THE INVENTION

The invention relates to a process for producing a fermentation product wherein the viscosity of the mash is reduced by application of beta-glucanase and xylanase activity.

### BACKGROUND OF THE INVENTION

Fermentation processes are used for making a vast number of products of commercial interest. Fermentation is used in industry to produce simple compounds such as alcohols (in particular ethanol); acids, such as citric acid, itaconic acid, lactic acid, gluconic acid, lysine; ketones; amino acids, such as glutamic acid, but also more complex compounds such as antibiotics, such as penicillin, tetracyclin; enzymes; vitamins, such as riboflavin, B₁₂, beta-carotene; hormones, such as insulin which are difficult to produce synthetically. Also in the brewing (beer and wine industry), dairy, leather, tobacco industries fermentation processes are used.

There is a large number of disclosures concerning production of fermentation products, e.g. ethanol, among which is WO2002038787A2.

There is a need for further improvement of fermentation processes and for improved processes including a fermentation step. Accordingly, the object of the invention is to provide an improved method of fermentation processes for producing e.g., ethanol.

### SUMMARY OF THE INVENTION

The present invention relates to an improved process of producing a fermentation product, in particular ethanol, but also for instance the products mentioned in the “Background of the Invention”-section. Also beverage production, such as beer production is contemplated according to the invention.

The invention provides in a first aspect a method of producing a fermentation product, said method comprising preliquefaction of non-starch polysaccharides in the presence of a beta-glucanase, followed by jet cooking and liquefaction in the presence of a thermostable beta-glucanase and a xylanase.

Provided in a second aspect is a method of producing a fermentation product, said method comprising the steps of: (a) providing a mash comprising a starch containing material and water; (b) preliquefying the mash of step (a) in the presence of a beta-glucanase; (c) gelatinizing the mash of step (b); (d) liquefying the mash of step (c) in the presence of a alpha-amylase and a beta-glucanase and a xylanase; and (e) saccharifying and fermenting the mash of step (d) to produce the fermentation product.

Provided in a second aspect is a use of beta-glucanase and xylanase in a process for producing ethanol.

The application of thinning enzymes such as beta-glucanase and xylanase in the process of the invention degrades glucan and xylan thereby reducing the viscosity of the mash. The reduced viscosity results in increased flow rates of the liquefied mash, thereby increasing the capacity of the production plants, especially by improving heat transfer and facilitating passage of the liquefied mash through the mash coolers. Thus the process of the invention facilitates the use of higher dry matter percentage in the fermentation while still securing an efficient cooling and a correct and uniform temperature of the mash delivered to the fermentation tanks.

The effect on the distillation process of the prior hydrolysis of non-starch polysaccharides like arabinoxylan and beta-glucans is an overall increased capacity and better heat transfer and phase transfer.

The effect on the by-products, such as the distiller's dry grain, of the prior hydrolysis of the non-starch polysaccharides is an overall improved feed conversion and better digestibility of the nutrients like minerals, protein, lipids and starch.

### DETAILED DESCRIPTION OF THE INVENTION

The process of the invention may be used in the production of a large number of fermentation products comprising but not limited to alcohols (in particular ethanol); acids, such as citric acid, itaconic acid, lactic acid, gluconic acid, lysine; ketones; amino acids, such as glutamic acid, but also more complex compounds such as antibiotics, such as penicillin, tetracyclin; enzymes; vitamins, such as riboflavin, B12, beta-carotene; hormones, such as insulin. Preferred is drinkable ethanol as well as industrial and fuel ethanol.

Raw Material

Any suitable starch containing material may be used as raw material in the process of the present invention. In one embodiment, the starch containing material is whole grain obtained from cereals, preferably selected from the list consisting of corn (maize), wheat, barley, oat, rice, cassava, sorghum, rye, milo, and millet. Furthermore the starch containing material may be obtained from potato, sweet potato, cassava, tapioca, sago, banana, sugar beet and/or sugar cane. Sugar cane or sugar beet may be utilized as described in e.g. GB 2115820 A and U.S. Pat. No. 4,886,672A1. Preferred for the process of the invention are cereals, such as wheat, barley, oat, triticale, especially oat and barley, as well as malt derived from cereals, such as wheat, barley, oat, triticale, especially oat and barley. Slurries made from wheat, barley, oat and triticale are highly viscous why thinning is advantageous.

Process Steps

The main process steps of the present invention may in one embodiment be described as separated into the following main process stages: (a) mash formation; (b) preliquefaction; (c) gelatnization; (d); liquefaction; and (e) saccharification and fermentation, wherein the steps (a), (b), (c) and (d) is performed in the order (a), (b), (c), (d) and (e). Step (e) may be performed as a simultaneous saccharification and fermentation (SSF) or as two separate sub steps.

The individual process steps of alcohol production may be performed batch wise or as a continuous flow. For the invention processes where all process steps are performed batch wise, or processes where all process steps are performed as a continuous flow, or processes where one or more process step(s) is(are) performed batch wise and one or more process step(s) is(are) performed as a continuous flow, are equally preferred.

The cascade process is an example of a process where one or more process step(s) is(are) performed as a continuous flow and as such preferred for the invention. For further information on the cascade process and other ethanol processes consult The Alcohol Textbook. Ethanol production by fermentation and distillation. Eds. T. P. Lyons, D. R. Kesall and J. E. Murtagh. Nottingham University Press 1995.

Milling

In a preferred embodiment of the process of the invention, the starch containing material is milled cereals, preferably barley, and the method comprises a step of milling the cereals before step (a). In other words, the invention also encompasses processes of the invention, wherein the starch containing material is obtainable by a process comprising milling of cereals, preferably dry milling, e.g. by hammer or roller mils. Grinding is also understood as milling, as is any process suitable for opening the individual grains and exposing the endosperm for further processing. Two processes of milling are normally used in alcohol production: wet and dry milling. The term “dry milling” denotes milling of the whole grain. In dry milling the whole kernel is milled and used in the remaining part of the process

Mash Formation

The mash may be provided by forming a slurry comprising the milled starch containing material and brewing water. The brewing water may be heated to a suitable temperature prior to being combined with the milled starch containing material in order to achieve a mash temperature of 45 to 70° C., preferably of 53 to 66° C., more preferably of 55 to 60° C. The mash is typically formed in a tank known as the slurry tank.

Typically the dry solids % (dry solid percentage) in the slurry tank (containing milled whole grain) is in the range from 1-60%, in particular 10-50%, such as 20-40%, such as 25-35%.

Preliquefaction

In the preliquefaction step the starch containing material (front end mash) is held in the presence of a thinning enzyme, such as a beta-glucanase or a xylanase, preferred are a beta-glucanase, at a temperature of 45 to 70° C., more preferably to 53 to 66° C., most preferably to 55 to 60° C., such as 58° C. The duration of the preliquefaction step is preferably 5 to 60 minutes, and more preferably 10 to 30 minutes, such as around 15 minutes.

Gelatinization

During the gelatinization step the starch is gelatinized. Gelatinization may be achived by heating the starch containing slurry to a temperature above the gelatinization temperature of the particular starch used. Gelatinization is preferably by jet-cooking at appropriate conditions, such as, e.g. at a temperature between 95-140° C., preferably 105-125° C., such as 120° C. to complete gelatinization of the starch. Also preferred is gelatinization by non-pressure cooking. During gelatinization the enzymes added in the preliquefaction step will be subjected to elevated temperatures and may be fully or partly inactivated. Thus according to the invention new thinning enzymes are preferably added following the gelatinization step.

The liquefaction process is in an embodiment carried out at pH 4.5-6.5, in particular at a pH between 5 and 6.

During jet cooking the enzymes added in the preliquefaction step will be subjected to elevated temperatures and may be fully or partly inactivated. Thus according to the invention new thinning enzymes are preferably added following the jet cooking step.

Liquefaction

In the liquefaction step the gelatinized starch (down stream mash) is broken down (hydrolyzed) into maltodextrins (dextrins). To achieve starch hydrolysis a suitable enzyme, preferably an alpha-amylase, is added.

According to the invention a beta-glucanase and a xylanase are added to the mash. In an embodiment further an endo-glucanase is added.

The temperature during the liquefaction step is from 60-95° C., preferably 80-90° C., preferably at 70-80° C. such as 85° C., for a period of 1-120 min, preferably for 2-60 min, such as 12 min. It is surprising that the enzymes functions at these high temperature applied during the liquefaction step.

In one embodiment, the liquefaction in step (d) is performed at a pH in the range of about pH 4-7, preferably pH about 4.5-6.5. In a preferred embodiment, the pH during the liquefaction is at most about 5. The pH of the slurry may by adjusted or not, depending on the properties of the enzymes used. Thus, in one embodiment the pH is adjusted, e.g. about 1 unit upwards, e.g. by adding NH₃. The adjusting of pH is advantageously done at the time when the alpha-amylase is added. In yet another embodiment, the pH is not adjusted and the alpha-amylase has a corresponding suitable pH-activity profile, such as being active at a pH about 4.

In an embodiment of the invention the thinning enzyme(s) is(are) added to the gelatinized mash together with an alpha-amylase.

Saccharification and Fermentation

The saccharification step and the fermentation step may be performed as separate process steps or as a simultaneous saccharification and fermentation (SSF) step. The saccharification is carried out in the presence of a saccharifying enzyme, e.g. a glucoamylase, a beta-amylase or maltogenic amylase. Optionally a phytase and/or a protease is added.

The fermenting organism may be a fungal organism, such as yeast, or bacteria. Suitable bacteria may e.g. be Zymomonas species, such as Zymomonas mobilis and E. coli. Examples of filamentous fungi include strains of Penicillium species. Preferred organisms for ethanol production are yeasts, such as e.g. Pichia or Saccharomyces. Preferred yeast according to the invention is Saccharomyces species, in particular Saccharomyces cerevisiae or bakers yeast. The yeast cells may be added in amounts of 10⁵ to 10¹², preferably from 10⁷ to 10¹⁰, especially 5×10⁷ viable yeast count per ml of fermentation broth. During the ethanol producing phase the yeast cell count should preferably be in the range from 10⁷ to 10¹⁰, especially around 2×10⁸. Further guidance in respect of using yeast for fermentation can be found in, e.g., “The alcohol Textbook” (Editors K. Jacques, T. P. Lyons and D. R. Kelsall, Nottingham University Press, United Kingdom 1999), which is hereby incorporated by reference

The microorganism used for the fermentation is added to the mash and the fermentation is ongoing until the desired amount of fermentation product is produced; in a preferred embodiment wherein the fermentation product is ethanol to be recovered this may, e.g., be for 24-96 hours, such as 35-60 hours. The temperature and pH during fermentation is at a temperature and pH suitable for the microorganism in question and with regard to the intended use of the fermentation product, such as, e.g., in an embodiment wherein the fermenting organism is yeast and the product is ethanol for recovery the preferred temperature is in the range about 26-34° C., e.g. about 32° C., and at a pH e.g. in the range about pH 3-6, e.g. about pH 4-5.

In another embodiment wherein the fermenting organism is yeast, and the fermented mash is to be used as a beer, the temperature of the mash the preferred temperature is around 12-16° C., such around 14° C.

In a preferred embodiment, a simultaneous saccharification and fermentation (SSF) process is employed where there is no holding stage for the saccharification, meaning that yeast and saccharification enzyme is added essentially together. In one embodiment, when doing SSF a pre-saccharification step at a temperature above 50° C. is introduced just prior to the fermentation.

Distillation

The method of the invention may further comprise recovering of the fermentation product, i.e. ethanol; hence the alcohol may be separated from the fermented material and purified.

Thus, in one embodiment, the method of the invention further comprises the step of: (f) distillation to obtain the ethanol,

By-Products from Distillation

The aqueous by-product (Whole Stillage) from the distillation process is separated into two fractions, for instance by centrifugation: 1) Wet Grain (solid phase), and 2) Thin Stillage (supernatant).

The Wet Grain fraction is dried, typically in a drum dryer. The dried product is referred to as “Distillers Dried Grain”, and can be used as animal feed.

The Thin Stillage fraction may be evaporated providing two fractions: —condensate fraction of 4-6% dry solids (mainly of starch, proteins, and cell wall components), and—syrup fraction, mainly consisting of limit dextrins and non fermentable sugars, which may be introduced into a dryer together with the Wet Grain (from the Whole Stillage separation step) to provide a product referred to as “Distillers Dried Grain”, which can be used as animal feed.

“Whole Stillage” is the term used in the art for the side-product coming from the distillation of fermented mash.

“Thin Stillage” is the term used in the art for the supernatant of the centrifugation of the Whole Stillage. Typically, the Thin Stillage contains 4-6% dry solids (mainly starch and proteins) and has a temperature of about 60-90° C. Thin Stillage is viscous and difficult to handle. Thin Stillage is normally kept in a holding tank for up to a few hours before recycling to the slurry tank. The stillage may be thinned with suitable enzymes, such as beta-glucanase and xylanase, before recycling.

Further details on how to carry out liquefaction, saccharification, fermentation, distillation, and recovering of ethanol are well known to the skilled person.

Use of the Products Produced by the Method of the Invention

In embodiments wherein the fermentation product is ethanol, the ethanol obtained by the process of the invention may be recovered from the fermented mash and used as, e.g., fuel ethanol; drinking ethanol, i.e., potable neutral spirits, or industrial ethanol, including fuel additive.

In embodiments wherein the fermentation product is ethanol, and the ethanol obtained by the process of the invention is not recovered from the fermented mash the mash comprising the ethanol may be used as a beer. The beer may be any beer including ales, strong ales, bitters, stouts, porters, lagers, export beers, malt liquors, barley wine, happoushu, high-alcohol beer, low-alcohol beer, low-calorie beer and light beer.

In embodiments wherein the fermentation product is not ethanol the product may be used for any suitable purpose.

Enzyme Activities

Beta-Glucanase (E.C. 3.2.1.4)

The beta-glucanase may be of microbial origin, such as derivable from a strain of a bacteria (e.g. Bacillus) or from a filamentous fungus (e.g., Aspergillus, Trichoderma, Humicola, Fusarium).

A beta-glucanases to be used in the processes of the invention may be an endo-glucanase, such as an endo-1,4-beta-glucanase. Commercially available beta-glucanase preparations which may be used include CELLUCLAST®, CELLUZYME®), CEREFLO® and ULTRAFLO® (available from Novozymes A/S), GC 880, LAMINEX™ and SPEZYME® CP (available from Genencor Int.) and ROHAMENT® 7069 W (available from Röhm, Germany). Preferred is CEREFLO®.

Beta-glucanases may be added in amounts of 0.01-5000 BGU/kg dry solids, preferably in the amounts of 0.1-500 BGU/kg dry solids, and most preferably from 1-50 BGU/kg dry solids and in the liquefaction step (down stream mash) in the amounts of 1.0-5000 BGU/kg dry solids, and most preferably from 10-500 BGU/kg dry solids.

Xylanase (EC 3.2.1.8 and other)

The process of the invention is carried out in the presence of an effective amount of a suitable xylanase which may be derived from a variety of organisms, including fungal and bacterial organisms, such as Aspergillus, Disporotrichum, Penicillium, Neurospora, Fusarum and Trichoderma.

Examples of suitable xylanases include xylanases derived from H. insolens (WO 92/17573; Aspergillus tubigensis (WO 92/01793); A. niger (Shei et al., 1985, Biotech. and Bioeng. Vol. XXVII, pp. 533-538, and Fournier et al., 1985, Bio-tech. Bioeng. Vol. XXVII, pp. 539-546; WO 91/19782 and EP 463 706); A. aculeatus (WO 94/21785).

The xylanase may also be a 1,3-beta-D-xylan xylanohydrolase (EC. 3.2.1.32).

In a specific embodiment the xylanase having is Xylanase II disclosed in WO 94/21785.

Contemplated commercially available compositions comprising xylanase include SHEARZYME® 200 L, SHEARZYME® 500 L, BIOFEED WHEAT®, and PULPZYME™ HC (from Novozymes) and GC 880, SPEZYME® CP (from Genencor Int).

Xylanases may be added in the amounts of 1.0-1000 FXU/kg dry solids, preferably from 5-500 FXU/kg dry solids, preferably from 5-100 FXU/kg dry solids and most preferably from 10-100 FXU/kg dry solids.

Alpha-Amylase(E.C. 3.2.1.1)

Preferred Alpha-Amylases are of Fungal or Bacterial Origin.

A Bacillus alpha-amylases (often referred to as “Termamyl-like alpha-amylases”), variant and hybrids thereof, are preferred according to the invention. Well-known Termamyl-like alpha-amylases include alpha-amylase derived from a strain of B. licheniformis (commercially available as Termamyl™), B. amyloliquefaciens, and B. stearothermophilus alpha-amylase. A suitable bacterial alpha-amylase may be the alpha-amylase derived from B. stearothermophilus and having the amino acid sequence disclosed as SEQ.NO:4 in W099/1 9467.

A suitable fungal alpha-amylases may be derived from Aspergillus, such as an acid fungal alpha-amylase derived from Aspergillus niger.

Commercial alpha-amylase products and products containing alpha-amylases include TERMAMYL™ SC, FUNGAMYL™, LIQUOZYME™ SC and SAN™ SUPER, (Novozymes A/S, Denmark) and DEX-LO™, SPEZYME™ AA, and SPEZYME™ DELTA AA (from Genencor Int.).

Fungal alpha-amylases may be added in the liquefaction step (d) in an amount of 0.001-1.0 AFAU/g dry solids, preferably from 0.002-0.5 AFAU/g dry solids, preferably 0.02-0.1 AFAU/g dry solids.

Bacillus alpha-amylases may be added in effective amounts well known to the person skilled in the art.

Maltogenic Amylase

The alph-amylase may be a maltogenic alpha-amylase. Maltogenic amylases (glucan 1,4alpha-maltohydrolase, E.C. 3.2.1.133) are able to hydrolyse amylose and amylopectin to maltose in the alpha-configuration. Furthermore, a maltogenic amylase is able to hydrolyse maltotriose as well as cyclodextrin. A specifically contemplated maltogenic amylase includes the one disclosed in EP patent no. 120,693 derived from Bacillus stearothermophilus C599. A commercially available maltogenic amylase is MALTOGENASE™ from Novozymes A/S

Glucoamylase

The saccharification step or the simultaneous saccharification and fermentation step (SSF) may be carried out in the presence of a glucoamylase. The glucoamylase may be of any origin, e.g. derived from a microorganism or a plant. Preferred is glucoamylase of fungal or bacterial origin selected from the group consisting of Aspergillus niger glucoamylase, in particular A. niger G1 or G2 glucoamylase (Boel et al. (1984), EMBO J. 3 (5), p. 1097-1102), or variants thereof, such as disclosed in WO 92/00381 and WO 00/04136; the A. awamori glucoamylase (WO 84/02921), A. oryzae (Agric. Biol. Chem. (1991), 55 (4), p. 941-949), or variants or fragments thereof.

Commercial products include SAN™ SUPER™ and AMG™ E (from Novozymes A/S). Glucoamylases may in an embodiment be added in the saccharification and fermentation step (e) in an amount of 0.02-2 AGU/g dry solids, preferably 0.1-1 AGU/g dry solids, such as 0.2 AGU/g dry solids.

Protease

Addition of protease(s) in the saccharification step, the SSF step and/or the fermentation step increase(s) the FAN (Free amino nitrogen) level and increase the rate of metabolism of the yeast and may increase the fermentation efficiency.

Suitable proteases include microbial proteases, such as fungal and bacterial proteases. Preferred proteases are acidic proteases, i.e., proteases characterized by the ability to hydrolyze proteins under acidic conditions below pH 7.

Suitable acid fungal proteases include fungal proteases derived from Aspergillus, Mucor, Rhizopus, Candida, Coriolus, Endothia, Enthomophtra, Irpex, Penicillium, Sclerotiumand Torulopsis. Especially contemplated are proteases derived from Aspergillus niger (see, e.g., Koaze et al., (1964), Agr. Biol. Chem. Japan, 28, 216), Aspergillus saitoi (see, e.g., Yoshida, (1954) J. Agr. Chem. Soc. Japan, 28, 66), Aspergillus awamori (Hayashida et al., (1977) Agric. Biol. Chem., 42(5), 927-933, Aspergillus aculeatus (WO 95/02044), or Aspergillus oryzae, such as the pepA protease; and acidic proteases from Mucor pusillus or Mucor miehei.

ALCALASE™ is a Bacillus licheniformis protease (subtilisin Carlsberg). ALCALASE™ may according to the invention preferably be added is amounts of 10⁻⁷to 10⁻³ gram active protease protein/g dry solids, in particular 10⁻⁶ to 10⁻⁴ gram active protease protein/g dry solids, or in amounts of 0.1-0.0001 AU/g dry solids, preferably 0.00025-0.001 AU/g dry solids.

FLAVOURZYME™ (available from Novozymes A/S) is a protease preparation derived from Aspergillus oryzae. FLAVOURZYME™ may according to the invention preferably be added in amounts of 0.01-1.0 LAPU/g dry solids, preferably 0.05-0.5 LAPU/g dry solids.

A suitable dosage of the protease is in the range in an amount of 10⁻⁷to 10⁻³ gram active protease protein/g dry solids, in particular 10⁻⁶ to 10⁻⁴ gram active protease protein/g dry solids

Phytase:

The phytase used according to the invention may be any enzyme capable of effecting the liberation of inorganic phosphate from phytic acid (myo-inositol hexakisphosphate) or from any salt thereof (phytates).

A suitable dosage of the phytase is in the range from 0.005-25 FYT/g dry solids, preferably 0.01-10 FYT/g, such as 0.1-1 FYT/g dry solids

Materials and Methods

Methods

Determination of Xylanase Activity (FXU)

The endoxylanase activity is determined by an assay, in which the xylanase sample is incubated with a remazol-xylan substrate (4-O-methyl-D-glucurono-D-xylan dyed with Remazol Brilliant Blue R. Fluka), pH 6.0. The incubation is performed at 50° C. for 30 min. The background of non-degraded dyed substrate is precipitated by ethanol. The remaining blue colour in the supernatant is determined spectrophotometrically at 585 nm and is proportional to the endoxylanase activity. The endoxylanase activity of the sample is determined relatively to an enzyme standard.

The assay is further described in the publication AF 293.6/1-GB, available upon request from Novo Nordisk A/S, Denmark.

Determination of Beta-Glucanase Activity (BGU)

The cellulytic activity may be measured in beta-glucanase units (BGU). Beta-glucanase reacts with beta-glucan to form glucose or reducing carbohydrate which is determined as reducing sugar using the Somogyi-Nelson method. 1 beta-glucanase unit (BGU) is the amount of enzyme which, under standard conditions, releases glucose or reducing carbohydrate with a reduction capacity equivalent to 1 μmol glucose per minute. Standard conditions are 0.5% beta-glucan as substrate at pH 7.5 and 30° C. for a reaction time of 30 minutes. A detailed description of the analytical method (EB-SM-0070.02/01) is available on request from Novozymes A/S.

Determination of Endo-Glucanase Activity (EGU)

The cellulytic activity may be measured in endo-glucanase units (EGU), determined at pH 6.0 with carboxymethyl cellulose (CMC) as substrate.

A substrate solution is prepared, containing 34.0 g/l CMC (Hercules 7 LFD) in 0.1 M phosphate buffer at pH 6.0. The enzyme sample to be analyzed is dissolved in the same buffer. 5 ml substrate solution and 0.15 ml enzyme solution are mixed and transferred to a vibration viscosimeter (e.g. MIVI 3000 from Sofraser, France), thermostated at 40° C. for 30 minutes.

One EGU is defined as the amount of enzyme that reduces the viscosity to one half under these conditions. The amount of enzyme sample should be adjusted to provide 0.01-0.02 EGU/ml in the reaction mixture. The arch standard is defined as 880 EGU/g.

Determination of Glucoamylase Activity (AGU)

The Novo Glucoamylase Unit (AGU) is defined as the amount of enzyme, which hydrolyzes 1 micromole maltose per minute at 37° C. and pH 4.3.

The activity is determined as AGU/ml by a method modified after (AEL-SM-0131, available on request from Novozymes) using the Glucose GOD-Perid kit from Boehringer Mannheim, 124036. Standard: AMG-standard, batch 7-1195, 195 AGU/ml. 375 microL substrate (1% maltose in 50 mM Sodium acetate, pH 4.3) is incubated 5 minutes at 37° C. 25 microL enzyme diluted in sodium acetate is added. The reaction is stopped after 10 minutes by adding 100 microL 0.25 M NaOH. 20 microL is transferred to a 96 well microtitre plate and 200 microL GOD-Perid solution (124036, Boehringer Mannheim) is added. After 30 minutes at room temperature, the absorbance is measured at 650 nm and the activity calculated in AGU/ml from the AMG-standard. A detailed description of the analytical method (AEL-SM-0131) is available on request from Novozymes.

Determination of Alpha-Amylase Activity (KNU)

The amylolytic activity may be determined using potato starch as substrate. This method is based on the break-down of modified potato starch by the enzyme, and the reaction is followed by mixing samples of the starch/enzyme solution with an iodine solution. Initially, a blackish-blue color is formed, but during the break-down of the starch the blue color gets weaker and gradually turns into a reddish-brown, which is compared to a colored glass standard.

One Kilo Novo alpha amylase Unit (KNU) is defined as the amount of enzyme which, under standard conditions (i.e. at 37° C.+/−0.05; 0.0003 M Ca²⁺; and pH 5.6) dextrinizes 5260 mg starch dry substance Merck Amylum solubile.

A folder EB-SM-0009.02/01 describing this analytical method in more detail is available upon request to Novozymes A/S, Denmark, which folder is hereby included by reference.

Determination of Protease (LAPU)

1 Leucine Amino Peptidase Unit (LAPU) is the amount of enzyme which decomposes 1 microM substrate per minute at the following conditions: 26 mM of L-leucine-p-nitroanilide as substrate, 0.1 M Tris buffer (pH 8.0), 40° C., 10 minutes reaction time.

### EXAMPLES

Enzymes used in the Examples:

A composition comprising beta-glucanase derived from Bacillus amyloliquefaciens; 1200 BGU/g.

A composition comprising Xylanase II disclosed in WO 94/21785 which is an endo 1-4 beta xylanase, derived from Aspergillus aculeatus; 521 FXU/g.

A composition derived from Trichoderma reesei comprising endo-glucanase activity and some xylanase and beta-glucanase activity; 700 EGU/g, 50 FXU/g, and 60 BGU/g.

A composition available from Genencor Int. as GC 880 “an engineered cellulase complex” comprising at least beta-glucanase and xylanase activity; 59 BGU/g and 222 FXU/g.

### Example 1

Front end slurry was prepared using 300 g milled barley flour in 700 ml of water in 1 liter flasks. pH was adjusted to 5.2 and the mash heated from room temperature (25° C.) to 54-60° C. in a temperature controlled water bath.

Different enzyme combinations were tested in dosages according to table 1. The viscosity was measured using a Haake Viscotester VT-02.

TABLE 1
Enzymes used in example 1, enzyme activity units per kg flour dry solids

| | FXU/kg | BGU/kg | EGU/kg |
|---|---|---|---|
| Beta-glucanase (B. amyloliquefaciens) | 38 | 346 | 0 |
| Xylanase II (A. Aculeatus) | | | |
| Beta-glucanase (B. amyloliquefaciens) | 0 | 432 | 0 |
| Beta-glucanase (B. amyloliquefaciens) | 21 | 348 | 30 |
| Xylanase II (A. Aculeatus) | | | |
| Endo-glucanase (T. reesei) | | | |
| GC 880 (Genencor Int) | 80 | 21 | n.a. |

n.a.; Not analysed

TABLE 2
Viscosity reduction using front end mash with different viscosity reducing
enzymes after 13, 26, 38 and 60 minutes

| | 13 min | 26 min | 38 min | 60 min |
|---|---|---|---|---|
| Beta-glucanase | 10 | 8 | 7 | 8 |
| (B. amyloliquefaciens) | | | | |
| Xylanase II (A. Aculeatus) | | | | |
| Beta-glucanase | 11 | 13 | 15 | 13 |
| (B. amyloliquefaciens) | | | | |
| Beta-glucanase | 11 | 8 | 7 | 8 |
| (B. amyloliquefaciens) | | | | |
| Xylanase II (A. Aculeatus) | | | | |
| Endo-glucanase | | | | |
| (Trichoderma reesei) | | | | |
| GC 880 (Genencor Int) | 17 | 15 | 15 | 14 |

The combinations of betaglucanase+xylanase II and betaglucanase+xylanase II+endo-glucanase resulted in a higher viscosity reduction that the product GC 880 or beta-glucanase alone.

### Example 2

Downstream viscosity reduction using the above mentioned non-starch degrading enzymes were tested in a slurry liquefied with bacterial alpha-amylase. The 28% dry solids slurry was DE 16 and pH 5.0. The slurry was portioned to 1 litre flasks and maintained at 84° C. in a temperature controlled water bath. Different enzyme combinations were tested in dosages according to table 3. The viscosity was measured as a function of time, using a Haake Viscotester VT-02, see table 4.

The combinations of betaglucanase+xylanase II+endo-glucanase worked more effectively that the product GC 880 or beta-glucanase alone or xylanase II+endo-glucanase.

TABLE 3
Enzymes used in example 2, enzyme activity units per kg flour dry solids.

| | FXU/kg | BGU/kg | EGU/kg |
|---|---|---|---|
| Beta-glucanase (B. amyloliquefaciens) | 31 | 288 | — |
| Xylanase II (A. Aculeatus) | | | |
| Beta-glucanase (B. amyloliquefaciens) | 35 | 292 | 42 |
| Xylanase II (Asp. Aculeatus) | | | |
| Endo-glucanase (T. reesei) | | | |
| Xylanase II (A. Aculeatus) | 87 | 9 | 105 |
| Endo-glucanase (T. reesei) | | | |
| GC 880 (Genencor) | 67 | 18 | n.a. |

n.a.; Not analysed

TABLE 4
Viscosity reduction during liquefaction at 84° C. with different viscosity
reducing enzymes after 4 minutes and after 10 minutes

| | 4 min | 10 min |
|---|---|---|
| Beta-glucanase (B. amyloliquefaciens) | 9 | 9.5 |
| Xylanase II (Asp. Aculeatus) | | |
| Endo-glucanase (Trichoderma reesei) | | |
| Beta-glucanase (B. amyloliquefaciens) | 11 | 10 |
| Xylanase II (Asp. Aculeatus) | | |
| Xylanase II (Asp. Aculeatus) | 11 | 10 |
| Endo-glucanase (Trichoderma reesei) | | |
| GC 880 (Genencor) | 13 | 13 |
| Blank (No extra enzyme) | 23 | 23 |

### Example 3

1 kg slurries of 30% grain dry matter were prepared by stirring milled rye into the corresponding amount of water, which had a temperature of 55° C. The pH was adjusted to 5.0 with sulphuric acid. The final temperature of the slurry was 50° C. Enzymes were added at t=0 minutes and mixed into the slurry by 3 minutes of stirring.

The viscosity was measured, using a Haake viscotester VT-02.

TABLE 5
Enzymes used in example 3, enzyme activity units per kg rye dry solids

| | FXU/kg | BGU/kg | EGU/kg |
|---|---|---|---|
| No enzyme | 0 | 0 | 0 |
| Beta-glucanase (B. amyloliquefaciens) | 78 | 150 | 0 |
| Xylanase II (A. Aculeatus) | | | |
| Beta-glucanase (B. amyloliquefaciens) | 0 | 450 | 0 |
| Xylanase II (A. Aculeatus) | 65 | 0 | 88 |
| Endo-glucanase (T. reesei) | | | |
| Xylanase II (A. Aculeatus) | 78 | 0 | 0 |

TABLE 6
Viscosity (dPa * S) using front end mash with different viscosity reducing
enzymes after 3, 15, 30 and 60 minutes

| | 3 min | 15 min | 30 min | 60 min |
|---|---|---|---|---|
| No enzyme | 200 | 100 | 75 | 60 |
| Beta-glucanase | 46 | 24 | 17 | 9 |
| (B. amyloliquefaciens) | | | | |
| Xylanase II (A. Aculeatus) | | | | |
| Beta-glucanase | 130 | 90 | 70 | 50 |
| (B. amyloliquefaciens) | | | | |
| Xylanase II (A. Aculeatus) | 48 | 32 | 26 | 18 |
| Endo-glucanase (T. reesei) | | | | |
| Xylanase II (A. Aculeatus) | 59 | 35 | 29 | 19 |

The combinations of betaglucanase+xylanase II and xylanase II+endo-glucanase resulted in a higher viscosity reduction that beta-glucanase or xylanase alone.

### Example 4

To simulate downstream processes in laboratory 1 kg slurries of 20% rye dry matter were prepared by stirring milled rye into the corresponding amount of water. Using a dosage of 0.5 kg Termamyl SC/tons of grain (as is) liquefaction was performed at 75° C. Afterwards the slurries were treated at 70° C. with viscosity reducing enzymes and measured at 70° C., pH=6-6.5. This was done as a model test in order to test the effect of the viscosity reducing enzymes. Industrially Termamyl+the non-starch polysaccharide degrading viscosity reducing enzymes should operate simultaneously.

The viscosity measurements were made using HAAKE Viscotester VT-02 was taken at t=3, 15, 30, 60 minutes as above, but at 70° C. The temperature was measured on the samples after the viscosity measurements.

TABLE 7
Enzymes used in example 4 (at 70° C.), enzyme
activity units per kg rye dry solids

| | FXU/kg | BGU/kg | EGU/kg |
|---|---|---|---|
| No enzyme | 0 | 0 | 0 |
| Beta-glucanase (B. amyloliquefaciens) | 98 | 76 | 0 |
| Xylanase II (A. Aculeatus) | | | |
| Xylanase II (A. Aculeatus) | 65 | 0 | 88 |
| Endo-glucanase (T. reesei) | | | |

TABLE 8
Viscosity in mPa * S using high temperature mashing (70° C.) at 20% dry
matter of rye (down stream) with different viscosity reducing
enzymes after 3, 15, 30 and 60 minutes

| | 3 min | 15 min | 30 min | 60 min |
|---|---|---|---|---|
| No enzyme | 200 | 170 | 190 | 260 |
| Beta-glucanase | 200 | 49 | 52 | 79 |
| (B. amyloliquefaciens) | | | | |
| Xylanase II (A. Aculeatus) | | | | |
| Xylanase II (A. Aculeatus) | 200 | 77 | 70 | 84 |
| Endo-glucanase (T. reesei) | | | | |

The combinations of beta-glucanase+xylanase II and endo-glucanase+xylanase II resulted in a high viscosity reduction at 70° C.

1-12. (canceled)

13. A method of producing ethanol, said method comprising the steps of:
a. providing a mash comprising a starch containing material and water;
b. preliquefying the mash of step (a) in the presence of a beta-glucanase;
c. gelatinizing the mash of step (b) by jet cooking;
d. liquefying the mash of step (c) in the presence of an alpha-amylase, a beta-glucanase and a xylanase; and
e. saccharifying and fermenting the mash of step (d) to produce ethanol.
f. recovering the ethanol.

14. The method of claim 13, further comprising a pre-saccharification step which is performed after the liquefaction step (d) and before step (e).

15. The method of claim 13, wherein the xylanase is derived from a strain of Aspergillus sp., preferably from a strain of A. Aculeatus.

16. The method of claim 13, wherein the beta-glucanase is derived from a strain of Bacillus sp., preferably from a strain of B. amyloliquefaciens.

17. The method of claim 13, wherein also an endo-glucanase is present in the liquefaction step (d), said endo-glucanase preferably derived from a strain of Trichoderma sp., preferably from a strain of T. reesei.

18. The method of claim 13, wherein the starch containing material is obtained from cereals and/or tubers.

19. The method of claim 13, wherein the starch containing material is selected from the groups consisting of maize, wheat, barley, rye, millet, sorghum, and milo.

20. The method of claim 13, wherein the starch containing material is selected from the groups consisting of potato, sweet potato, cassava, tapioca, sago, banana, sugar beet and sugar cane.

21. The method of claim 13, wherein the fermentation in step (e) is performed using a micro-organism, such as bacteria and fungi (including yeasts), e.g. Zymomonas species and Sacharomyces species such as e.g. Saccharomyces cerevisiae.

22. The method of claim 13, wherein the fermentation is carried out in the presence of phytase and/or protease.

23. The method of claim 13, wherein preliquefaction in step (b) is performed at a temperature of 45 to 70° C., of 53 to 66° C., of 55 to 60° C., of 58° C. for a period of 5 to 60 minutes, and of 10 to 30 minutes, around 15 minutes.

24. The method of claim 13, wherein the liquefaction in step (d) is performed at 60-95° C., at 80-90 □C for 10-120 min, at 83-85 □C for 15-80 min.
